(19) United States
(12) Patent Application Publication
(10) Pub. No.: US 2024/0350380 A1
(43) Pub. Date: Oct. 24, 2024

(54) Mineral Sunscreen Compositions

(71) Applicant: Johnson & Johnson Consumer Inc., Skillman, NJ (US)

(72) Inventors: Jeffrey D. Martin, Lawrenceville, NJ (US); Katherine Skinner, Navarre, FL (US); Frank C. Sun, Belle Mead, NJ (US)

(21) Appl. No.: 18/136,456

(22) Filed: Apr. 19, 2023

(51) Int. Cl. A61K 8/27; A61K 8/29; A61K 8/31; A61K 8/85; A61K 8/92; A61Q 17/04

(52) U.S. Cl. CPC A61K 8/27; A61K 8/29; A61K 8/31; A61K 8/85; A61K 8/922; A61Q 17/04

(57) ABSTRACT

The present invention provides an anhydrous sunscreen composition comprising: (a) at least about 15% by weight based on the total weight of the sunscreen composition of a combination of titanium dioxide and zinc oxide, wherein the weight ratio of zinc oxide to titanium dioxide in the composition is about 0.5 to about 1.5; (b) at least about 50% by weight based on the total weight of the sunscreen composition of a carrier oil, wherein the carrier oil comprises at least 50% by weight Helianthus Annuus (Sunflower) Seed Oil; (c) about 10% to about 20% by weight based on the total weight of the sunscreen composition a combination of Isododecane and Diisopropyl Adipate; and (d) about 2% to about 6% by weight based on the total weight of the sunscreen composition of a film former comprising triacontanyl PVP.

The present invention provides anhydrous sunscreen compositions containing a specific combination of mineral UV blockers, carrier, emollients and film formers. They do not require the use of organic UV absorbers, but still desirably provide high SPF products that are stable and optionally sprayable.

### BACKGROUND OF THE INVENTION

It is well known that prolonged exposure to ultraviolet radiation, such as from the sun, can lead to the formation of light dermatoses and erythemas, and increase the risk of skin cancers, such as melanoma. Exposure to UV radiation also accelerates skin aging, such as loss of skin elasticity and wrinkling.

For these reasons, sunscreen compositions are commonly used to provide protection from the sun, and a variety of sunscreen compositions are commercially available. Many contain organic UV absorbers such as octocrylene, homosalate, avobenzone, oxybenzone, octyl salicylate and cinnamates. While these UV filters are effective for absorbing UV radiation, high amounts are often required to provide a high SPF sunscreen composition. Because they are organic compounds, consumers are sometimes concerned about their potential to penetrate the skin, particularly when used in high amounts.

Improved sunscreen compositions substantially free of organic UV absorbers and therefore substantially non-penetrating to the skin, have now been discovered. Such compositions contain a specific combination of mineral UV blockers, carrier, emollients and film formers, but still desirably provide high SPF products that are stable and optionally sprayable.

### SUMMARY OF THE INVENTION

The present invention provides anhydrous sunscreen composition comprising: (a) at least about 15% by weight based on the total weight of the sunscreen composition of a combination of titanium dioxide and zinc oxide, wherein the weight ratio of zinc oxide to titanium dioxide in the composition is about 0.5 to about 1.5; (b) at least about 50% by weight based on the total weight of the sunscreen composition of a carrier oil, wherein the carrier oil comprises at least 50% by weight Helianthus Annuus (Sunflower) Seed Oil; (c) about 10% to about 20% by weight based on the total weight of the sunscreen composition a combination of Isododecane and Diisopropyl Adipate; and (d) about 2% to about 6% by weight based on the total weight of the sunscreen composition of a film former comprising Triacontanyl PVP.

### DETAILED DESCRIPTION OF THE INVENTION

Unless defined otherwise, all technical and scientific terms used herein have the same meaning as commonly understood by one of ordinary skill in the art to which the invention belongs. All publications, patent applications, patents, and other references mentioned herein are incorporated by reference.

As used herein, “topically applying” means directly laying on or spreading on outer skin or the scalp, e.g., by use of the hands or an applicator such as a wipe, roller, or spray.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin.

As used herein, “sunscreen composition” means a formulation (e.g., a lotion, spray, gel or other topical product) that absorbs and/or reflects some of the sun's ultraviolet (UV) radiation and thus helps protect against negative effects of sun exposure, e.g., sunburn, premature aging, etc.

As used herein, “cosmetically effective amount” means an amount of a physiologically active compound or composition sufficient for treating one or more conditions, but low enough to avoid serious side effects. The cosmetically effective amount of the compound or composition will vary with the condition being treated, the age and physical condition of the end user, the severity of the condition being treated/prevented, the duration of the treatment, the nature of other treatments, the specific compound or product/composition employed, the cosmetically-acceptable carrier utilized, and like factors.

As used herein, “cosmetically acceptable” means that the ingredients the term describes are suitable for use in contact with tissues (e.g., the skin) without undue toxicity, incompatibility, instability, irritation, allergic response, or the like.

As used herein, a “cosmetically acceptable active agent” is a compound (synthetic or natural) that has a cosmetic or therapeutic effect on the skin.

As used herein, “treatment or treating” refers to mitigating, reducing, preventing, improving, or eliminating the presence or signs of a condition or disorder.

As used herein, “substantially free of” means the ingredient referred to is not directly or intentionally added. Preferably, “substantially free of” means containing less than about 1% by weight. More preferably “substantially free of” means containing less than about 0.5% by weight. Even more preferably “substantially free of” means containing less than about 0.1% by weight. The present sunscreen composition may be completely free of an ingredient, i.e., contain none of the ingredient.

As used herein, “UV absorber” means a compound that absorbs radiation in some portion of the ultraviolet spectrum (290 nm-400 nm), such as one having an extinction coefficient of at least about 1000 mol⁻¹ cm⁻¹, for at least one wavelength within the above-defined ultraviolet spectrum.

As used herein, “UV blocker” means a compound that reflects or scatters UV radiation.

Unless otherwise indicated, a percentage or concentration refers to a percentage or concentration by weight (i.e., % (W/W). Unless stated otherwise, all ranges are inclusive of the endpoints, e.g., “from 4 to 9” includes the endpoints 4 and 9.

### Mineral UV Blockers

The sunscreen composition of the invention comprises a combination of mineral UV blockers, specifically titanium dioxide and zinc oxide.

The composition comprises at least about 15% by weight of the combination of titanium dioxide and zinc oxide. Preferably, the composition comprises at least about 20% by weight of the combination of titanium dioxide and zinc oxide. More preferably, the composition comprises at least about 24% by weight of the combination of titanium dioxide and zinc oxide. The composition may contain about 24% by weight of the combination of titanium dioxide and zinc oxide. The composition may contain about 25% by weight of the combination of titanium dioxide and zinc oxide.

The weight ratio of zinc oxide to titanium dioxide in the composition ranges from about 0.5 to about 1.5. The weight ratio of zinc oxide to titanium dioxide in the composition may be about 0.67 to about 1.4. The weight ratio of zinc oxide to titanium dioxide in the composition may be about 0.67. The weight ratio of zinc oxide to titanium dioxide in the composition may be about 1. The weight ratio of zinc oxide to titanium dioxide in the composition may be about 1.4. The weight ratio of zinc oxide to titanium dioxide in the composition may be about 0.67.

An example of a titanium dioxide that may be used is Titanium Dioxide; Aluminum Hydroxide; Stearic Acid sold under the trade name, MICRO TITANIUM DIOXIDE MT-100TV by Tayca Corporation.

An example of a zinc oxide that may be used is Zinc Oxide; Triethoxycaprylylsilane sold under the trade name, Micro Zinc Oxide MZX-304OTS by Tayca Corporation.

Preferably, the composition is substantially free of UV absorbers. In particular, the composition may be substantially free of low molecular weight, organic UV absorbers. As used herein, “low molecular weight, organic UV absorber” means an organic molecule capable of absorbing UV light, typically an aromatic compound conjugated with a carbonyl moiety substituted in the ortho- or para-position of the aromatic ring and having a molecular weight of less than 900 g/mol.

Preferably, the composition is completely free of low molecular weight, organic UV absorbers.

Examples of low molecular weight, organic UV absorbers include, but are not limited to: methoxycinnamate derivatives such as octyl methoxycinnamate and isoamyl methoxycinnamate; camphor derivatives such as 4-methyl benzylidene camphor, camphor benzalkonium methosulfate, and terephthalylidene dicamphor sulfonic acid; salicylate derivatives such as octyl salicylate, ethylhexyl salicylate and homosalate; benzone derivatives such as dioxybenzone, and oxybenzone; benzoic acid derivatives such as aminobenzoic acid and octyldimethyl para-amino benzoic acid; octocrylene and other β,β-diphenylacrylates; dioctyl butamido triazone; octyl triazone; avobenzone (butyl methoxydibenzoylmethane); menthyl anthranilate; triazone derivatives such as ethylhexyl triazone (Uvinul® T150); diethylhexyl butamido triazone (UVASorb® HEB); bis-ethylhexyloxyphenol methoxyphenyl triazine (Tinosorb® S), benzoate derivatives such as diethylamino hydroxybenzoyl hexyl benzoate (Uvinul® A Plus), benzotriazole derivatives such as drometrizole trisiloxane (Mexoryl® XL), methylene bis-benzotriazolyl tetramethylbutylphenol (Tinosorb® M); tris-biphenyl triazine; (2-{4-[2-(4-Diethylamino-2-hydroxy-benzoyl)-benzoyl]-piperazine-1-carbonyl}-phenyl)-(4-diethylamino-2-hydroxy-phenyl)-methanone; merocyanine derivatives; bis(butylbenzoate) diaminotriazine aminopropylsiloxane; and bis-ethylhexyloxyphenol methoxyphenyl triazine encapsulated in a polymer matrix.

The composition may in certain embodiments also be free of polymeric organic UV absorbers. As used herein, “polymeric organic UV absorber” means a polymeric compound comprising organic chromophores attached to a polymer chain. For instance, the polymer chain may be a polysiloxane having for example an average molecular weight of greater than 6000 Daltons. Examples of such polysiloxane UV absorbers include, without limitation, Parsol® SLX commercially available from DSM and polysilicone-15. These polysiloxanes absorb in the UVB (Amax=312 nm) part of the spectrum and are typically combined with UVA filters to achieve broad-spectrum protection.

Alternatively, the composition may contain one or more polymeric organic UV absorbers. Because of their relatively high molecular weight, they are regarded as substantially non-absorbing to the skin.

### Carrier Oil

The sunscreen composition comprises at least about 50% by weight based on the total weight of the composition of a carrier oil. The composition may contain about 55 to about 60% by weight based on the total weight of the composition of a carrier oil. The composition may contain about 55.5% by weight based on the total weight of the composition of a carrier oil. The composition may contain about 58.5% by weight based on the total weight of the composition of a carrier oil.

At least 50% by weight of the carrier oil is Helianthus Annuus (Sunflower) Seed Oil. Optionally, the carrier oil may comprise at least 80% by weight Helianthus Annuus (Sunflower) Seed Oil.

Helianthus Annuus (Sunflower) Seed Oil is commercially available, for example, as Refined and Winterised sunflower oil from SAIPOL, 101 route de bourbourg BP 89 FR-59210 COUDEKERQUE BRANCHE and Sunflower Oil Refined (TX008081) from Textron Tecnica.

Helianthus Annuus (Sunflower) Seed Oil may be the only ingredient in the carrier oil, or alternatively the carrier oil may comprise ingredients in addition to Helianthus Annuus (Sunflower) Seed Oil, provided the Helianthus Annuus (Sunflower) Seed Oil is 50% by weight or greater of the carrier oil.

In one embodiment, the carrier oil comprises Helianthus Annuus (Sunflower) Seed Oil and Caprylic/Capric Triglyceride.

### Isododecane and Diisopropyl Adipate

The composition also comprises about 10% to about 20% by weight based on the total weight of the sunscreen composition a combination of Isododecane and Diisopropyl Adipate. The composition may comprise about 13% by weight based on the total weight of the sunscreen composition a combination of Isododecane and Diisopropyl Adipate.

Preferably the combination comprises a greater amount of Isododecane than Diisopropyl Adipate. For example, the weight ratio of Isododecane to Diisopropyl Adipate in the composition may be at least about 1.25, preferably at least about 1.5, for example 1.6.

Isododecane and Diisopropyl Adipate are known emollients for skin care products.

Isododecane for example is commercially available from INEOS Manufacturing Deutschland GmbH Koln Germany under the trade name Permethyl 99A.

Diisopropyl Adipate for example is commercially available from Lubrizol Advanced Materials under the trade name, Schercemol™ DIA ester.

### Film Former

The composition further comprises about 2% to about 6% by weight based on the total weight of the sunscreen composition of a film former comprising Triacontanyl PVP.

The film former may comprise only Triacontanyl PVP or optionally contain additional compounds. For example, the composition may comprise a combination of Triacontanyl PVP and VP/Hexadecene Copolymer.

Triacontanyl PVP is a known film former, and commercially available for example from ISP FREETOWN FINE CHEMICALS INC under the trade name Antaron™ WP-660 polymer.

In one embodiment, the composition comprises about 2.5% to about 3.2% by weight based on the total weight of the sunscreen composition of Triacontanyl PVP and about 2.5% by weight based on the total weight of the sunscreen composition of VP/Hexadecene Copolymer.

### SPF

Sun protection factor (SPF) of the composition may be tested using the following IN VITRO SPF TEST METHOD. The baseline transmission of a PMMA plate (substrate, available from Helioscience, Marseille, France) is measured for UV absorbance using calibrated Labsphere® UV-10005 UV transmission analyzer or a Labsphere® UV-2000S UV transmission analyzer (Labsphere, North Sutton, N.H., USA). A test sample is then applied to the PMMA plate using an application density of about 1.3 mg/cm2 by rubbing into a uniform thin layer with the operator's finger. The sample is allowed to dry for 15 minutes and then measured for UV absorbance in the same way. The absorbance measures are used to calculate SPF as known in the art using the following equation:

$$SPF in vitro = \frac{{\int }_{λ = 290 nm}^{λ = 400 nm} E ( λ ) * I ( λ ) * d λ}{{\int }_{λ = 290 nm}^{λ = 400 nm} E ( λ ) * I ( λ ) * {10}^{{A}_{0} ( λ )} * d λ}$$

in which:
E(λ)=Erythema action spectrum;
I(λ)=Spectral irradiance received from the UV source;
A₀(λ)=Mean monochromatic absorbance of the test product layer before UV exposure; and
d(λ)=Wavelength step (1 nm).

In one embodiment, the composition has an SPF as measured by the IN VITRO SPF TEST METHOD of at least about 15. In another embodiment, the composition has an SPF as measured by the IN VITRO SPF TEST METHOD of at least about 25. In a further embodiment, the composition has an SPF as measured by the IN VITRO SPF TEST METHOD of at least about 35.

### Topical Composition

The sunscreen composition is anhydrous. The composition may be substantially free of water. The composition may be completely free of water.

The composition may be prepared using mixing and blending methodology well known in the sunscreen and cosmetic art.

The composition may be in the form or a serum, for example dispensed using a dropper.

The composition may be in the form of a spray, as further described below.

The composition may be combined with a “cosmetically-acceptable topical carrier,” i.e., a carrier for topical use that capable of containing the other ingredients dispersed or dissolved therein and possessing acceptable properties rendering it safe to use topically.

The cosmetically-acceptable topical carrier may optionally comprise a wide variety of additional oil-soluble materials and/or oil-dispersible materials conventionally used in compositions for use on skin, at their art-established levels. For example, surfactants, emulsifiers, pearlescent or opacifying agents, thickeners, emollients, conditioners, humectants, chelating agents, exfoliants, preservatives, pH adjusting agents, dispersing agents, and additives that enhance the appearance, feel, or scent of the composition, such as colorants, fragrances, tactile modifiers, and the like, can be included.

The composition may optionally comprise additional film formers for instance natural polymers such as polysaccharides or proteins and synthetic polymers such as other polyesters, polyacrylics, polyurethanes, vinyl polymers, polysulfonates, polyureas, polyoxazolines, and the like. Specific examples include acrylates/dimethicone acrylate copolymer (commercially available as X-22-8247D from Shin-Etsu of Japan); and hydrogenated dimer dilinoleyl/dimethylcarbonate copolymer (commercially available from BASF Corp. as COSMEDIA DC). The composition may optionally contain additional emollients including mineral oils, petrolatum, vegetable oils, waxes, and esters, including but not limited to isopropyl palmitate, isopropyl myristate, dibutyl adipate, dicaprylyl carbonate, C12-15 alkyl benzoate), silicone oils such as dimethicone, and alkanes.

In certain embodiments, the composition includes a pigment suitable for providing color or hiding power. The pigment may be one suitable for use in a color cosmetic product, including compositions for application to the hair, nails and/or skin, especially the face. Color cosmetic compositions include, but are not limited to, foundations, concealers, primers, blush, mascara, eyeshadow, eyeliner, lipstick, nail polish and tinted moisturizers. The pigment suitable for providing color or hiding power may be composed of iron oxides, including red and yellow iron oxides, titanium dioxide, ultramarine and chromium or chromium hydroxide colors, and mixtures thereof. The pigment may be a lake pigment, e.g. an organic dye such as azo, indigoid, triphenylmethane, anthraquinone, and xanthine dyes that are designated as D&C and FD&C blues, browns, greens, oranges, reds, yellows, etc., precipitated onto inert binders such as insoluble salts. Examples of lake pigments include Red #6, Red #7, Yellow #5, Violet #2 and Blue #1. The pigment may be an interference pigment. Examples of interference pigments include those containing mica substrates, bismuth oxycloride substrates, and silica substrates, for instance mica/bismuth oxychloride/iron oxide pigments commercially available as CHROMALITE pigments (BASF), titanium dioxide and/or iron oxides coated onto mica such as commercially available FLAMENCO pigments (BASF), mica/titanium dioxide/iron oxide pigments including commercially available KTZ pigments (Kobo products), CELLINI pearl pigments (BASF), and borosilicate-containing pigments such as REFLECKS pigments (BASF).

In one embodiment, the composition comprises one or more SPF boosters, such as styrene/acrylates copolymer. A commercially available styrene/acrylates copolymer is SUNSPHERES Powder from Dow Chemical.

The composition may comprise one or more dispersing agents, such as Polyhydroxystearic Acid. For example, the composition may comprise about 1 to about 2% by weight based on the total weight of the composition of Polyhydroxystearic Acid. Or the composition may comprise about 1.5% by weight based on the total weight of the composition of Polyhydroxystearic Acid.

In another embodiment, the composition is substantially free of polyester-7. Polyester-7 is a copolymer of trimethylolpropane, adipic acid, neopentyl glycol and hexanediol (and) neopentyl glycol diheptanoate and commercially available for example as LEXFILM SUN.

The composition may further comprise one or more other cosmetically acceptable active agents include for example anti-acne agents, shine control agents, anti-microbial agents, anti-inflammatory agents, anti-mycotic agents, anti-parasite agents, external analgesics, antioxidants, keratolytic agents, moisturizers, nutrients, vitamins, energy enhancers, anti-perspiration agents, astringents, deodorants, firming agents, anti-callous agents, and agents for skin conditioning.

The amount of other cosmetically active agents may range from about 0.001% to about 20% by weight of the composition, e.g., about 0.005% to about 10% by weight of the composition, such as about 0.01% to about 5% by weight of the composition.

The cosmetically acceptable active agent may be selected for instance from D-panthenol carotenoids, ceramides, polyunsaturated fatty acids, essential fatty acids, enzymes such as laccase, enzyme inhibitors, minerals, steroids such as hydrocortisone, 2-dimethylaminoethanol, copper salts such as copper chloride, peptides like argireline, syn-ake and those containing copper, coenzyme Q10, amino acids such as proline, vitamins, lactobionic acid, acetyl-coenzyme A, niacin, riboflavin, thiamin, ribose, electron transporters such as NADH and FADH2, natural extracts such as from aloe vera, feverfew, oatmeal, dill, blackberry, princess tree, Picia anomala, and chicory, resorcinols such as 4-hexyl resorcinol, curcuminoids, sugar amines such as N-acetyl glucosamines, and derivatives and mixtures thereof.

Examples of vitamins include, but are not limited to, vitamin A, vitamin B's such as vitamin B3, vitamin B5, and vitamin B12, vitamin C, vitamin K, and different forms of vitamin E like alpha, beta, gamma or delta tocopherols or their mixtures, and derivatives thereof.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, propolis and Zingiber Officinale (Ginger) Root Extract.

In one embodiment, the composition comprises one or more of tocopheryl acetate, ascorbyl palmitate/tocopherol/Lecithin, and Zingiber Officinale (Ginger) root extract as antioxidants. All three of the foregoing may be used together.

### Stability

The sunscreen compositions of the invention are advantageously stable. As used herein, “stability” means having the “Successful Prototype” for “Separation” and “Sedimentation” set forth in Table 1.

TABLE 1
Definition of Stability

| | Successful Prototype |
|---|---|
| Separation | Room temperature sample having |
| | 1 inch or less of separation at |
| | any time up to 1 month aging. |
| Sedimentation | Room temperature sample that |
| | exhibits no sedimentation after gently |
| | shaking the sample 5-10 times, |
| | at any time up to 1 month aging. |

### Sprayability

In certain embodiments, the sunscreen composition is also sprayable. “Sprayable” as used herein means the composition, when manually actuated or through pressurized release out of a dispensing mechanism, such as a bottle with pump spray nozzle or an aerosol can, creates a spray pattern evenly distributed and reproducible over an area of a defined shape (e.g. circle, annulus) and size. The composition may be sprayable without the use of propellants, i.e., in non-aerosol form.

Sprayable sunscreen compositions according to the invention may have shear viscosities of less than about 300, preferably less than about 200, more preferably less than about 180, cP at 1,000 s⁻¹ at 25° C. Shear viscosity is defined as a fluid's resistance to a shearing flow and is given in units of centipoise (cP). Shear viscosity of the sunscreen compositions is measured using the SHEAR VISCOSITY TEST METHOD below.

### Shear Viscosity Test Method

Shear viscosity is measured by the following method. An appropriate amount of sample is loaded into an Anton Paar MCR302 rheometer equipped with a double-wall Couette geometry. Temperature is held at 25° C. by a Peltier element, and the shear rate is ramped from 1,000 to 0.1 s⁻¹ using the default steady-state criteria in the rheometer control software RheoCompass.

The following non-limiting examples further illustrate the invention.

### Example 1

The following Compositions E1-E6 according to the invention were made using the ingredients shown in Table 2.

In vitro SPF was tested on Compositions E1, E2, E3 and E5 using the IN VITRO SPF TEST METHOD set forth above. The results are also shown in Table 2.

TABLE 2

| | | Weight (%) | | | | | |
|---|---|---|---|---|---|---|---|
| INCI Name | Function | E1 | E2 | E3 | E4 | E5 | E6 |
| Helianthus Annuus | Carrier Oil | 57.3 | 55.5 | 27.856 | 56.096 | 58 | 58.496 |
| (Sunflower) Seed | | | | | | | |
| Oil | | | | | | | |
| Caprylic/Capric | Carrier Oil | 0 | 0 | 27.74 | 0 | 0 | 0 |
| Triglyceride | | | | | | | |
| Isododecane | Emollient | 8 | 8 | 8 | 8 | 8 | 8 |
| Diisopropyl Adipate | Emollient | 5 | 5 | 5 | 5 | 5 | 5 |
| VP/Hexadecene | Film | 0 | 2.5 | 2.5 | 2.5 | 0 | 0 |
| Copolymer | Former | | | | | | |
| Triacontanyl PVP | Film | 3.2 | 2.5 | 2.5 | 2.5 | 2.5 | 2.5 |
| | Former | | | | | | |
| Tocopheryl Acetate | Antioxidant | 0 | 0 | 0.5 | 0.4 | 0 | 0.5 |
| Ascorbyl Palmitate; | Antioxidant | 0 | 0 | 0.004 | 0.004 | 0 | 0.004 |
| Tocopherol; | | | | | | | |
| Lecithin | | | | | | | |
| Zingiber Officinale | Antioxidant | 0 | 0 | 0.2 | 0 | 0 | 0 |
| (Ginger) Root | | | | | | | |
| Extract | | | | | | | |
| Fragrance | Fragrance | 0 | 0 | 0.2 | 0 | 0 | 0 |
| Polyhydroxystearic | Dispersing | 1.5 | 1.5 | 1.5 | 1.5 | 1.5 | 1.5 |
| Acid | Agent | | | | | | |
| Zinc Oxide; | Sunscreen | 10 | 10 | 12 | 14 | 10 | 12 |
| Triethoxycaprylylsilane | Agent | | | | | | |
| Titanium | Sunscreen | 15 | 15 | 12 | 10 | 15 | 12 |
| Dioxide; Aluminum | Agent | | | | | | |
| Hydroxide; Stearic | | | | | | | |
| Acid | | | | | | | |

| | | | | | | |
|---|---|---|---|---|---|---|
| In vitro SPF | 116.29 | 57.57 and 80.61 | 35.256 | Not | 112.72 | Not |

| | |
|---|---|
| tested | tested |

Compositions E1-E6 were prepared by adding the carrier oil(s) to a glass beaker at room temperature and placing that beaker on a hot plate. A propeller was lowered into the beaker and turned on to such a speed to form a vortex. To the glass beaker Isododecane and Diisopropyl Adipate were added. Next Polyhydroxystearic Acid and VP/Hexadecene Copolymer, if applicable, were added to the glass beaker and mixed well. Heating to 70-75° C. was initiated. Once temperature was in range, Triacontanyl PVP was added and mixed until dispersed into the solution. A check was performed to ensure there were no undispersed particles in the glass beaker. Next Zinc Oxide; Triethoxycaprylylsilane was slowly added to the beaker and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Cooling to 35-45° C. was initiated. If applicable Tocopheryl Acetate was then added to the beaker and mixed. If applicable, Ascorbyl Palmitate; Tocopherol; Lecithin, was then added to the beaker and mixed. Mixing with the propeller was stopped and the formula was homogenized with a SILVERSON Benchtop Homogenizer at 3,500-4,000 RPM for 2-4 minutes depending on the batch size.

Compositions E1 through E6 were stable according to the Successful Prototype definition in Table 1. In addition, they also exhibited stability (less than 1 inch of separation and no sedimentation) for 1 month at 40° C. and 1 week at 50° C.

### Example 2

The following Comparative Compositions A-F were made using the ingredients shown in Table 3.

In vitro SPF was tested on all the compositions using the IN VITRO SPF TEST METHOD set forth above. The results are also shown in Table 3.

TABLE 3

| | | Weight (%) | | | | | |
|---|---|---|---|---|---|---|---|
| INCI Name | Function | A | B | C | D | E | F |
| Helianthus Annuus | Carrier Oil | 59.5 | 59.5 | 58.496 | 58.5 | 54 | 56.5 |
| (Sunflower Seed Oil) | | | | | | | |
| Isododecane | Emollient | 8 | 8 | 8 | 8 | 12 | 8 |
| Diisopropyl Adipate | Emollient | 5 | 5 | 5 | 5 | 3 | 5 |
| VP/Hexadecene | Film Former | 0 | 1 | 2.5 | 1 | 0 | 2.5 |
| Copolymer | | | | | | | |
| Triacontanyl PVP | Film Former | 0 | 0 | 0 | 0 | 0 | 1.5 |
| Polyester-7 (and) | Film Former | 1 | 0 | 0 | 1 | 0 | 0 |
| Neopentyl Glycol | | | | | | | |
| Diheptanoate | | | | | | | |
| Polyamide-8 | Film Former | 0 | 0 | 0 | 0 | 5 | 0 |
| Ascorbyl Palmitate; | Antioxidant | 0 | 0 | 0.004 | 0 | 0 | 0 |
| Tocopherol; Lecithin | | | | | | | |
| Tocopheryl Acetate | Antioxidant | 0 | 0 | 0.5 | 0 | 0 | 0 |
| Polyhydroxystearic Acid | Dispersing | 1.5 | 1.5 | 1.5 | 1.5 | 1 | 1.5 |
| | Agent | | | | | | |
| Zinc Oxide; | Sunscreen | 10 | 10 | 12 | 10 | 10 | 10 |
| Triethoxycaprylylsilane | Agent | | | | | | |
| Titanium Dioxide; | Sunscreen | 15 | 15 | 12 | 15 | 15 | 15 |
| Aluminum Hydroxide; | Agent | | | | | | |
| Stearic Acid | | | | | | | |

| | | | | | | |
|---|---|---|---|---|---|---|
| In vitro SPF | 17.65 | 13.086 | 17.356 | 19.79 | 78.845 | 47.79 |

Comparative Compositions A-F were prepared by adding the carrier oil to a glass beaker at room temperature and placing that beaker on a hot plate. A propeller was lowered into the beaker and turned on to such a speed to form a vortex. To the glass beaker Isododecane and Diisopropyl Adipate were added. Next Polyhydroxystearic Acid was added to the glass beaker and mixed well.

For Comparative Compositions A and D, Zinc Oxide; Triethoxycaprylylsilane was next slowly added to the beaker at room temperature and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Polyester-7 (and) Neopentyl Glycol Diheptanoate was next added to the beaker and mixed. For Comparative Composition D, VP/Hexadecene Copolymer was next added to the beaker and mixed until homogenous. Mixing with the propeller was stopped and the formula was homogenized with a SILVERSON Benchtop Homogenizer at 4,000 RPM for 2.5 minutes.

For Comparative Composition B, Zinc Oxide; Triethoxycaprylylsilane was slowly added to the beaker at room temperature and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. VP/Hexadecene Copolymer was next added to the beaker and mixed until homogenous. Mixing with the propeller was stopped and the formula was homogenized with a SILVERSON Benchtop Homogenizer at 4,000 RPM for 2.5 minutes.

For Comparative Composition C, VP/Hexadecene Copolymer was added to the beaker and mixed until homogenous. Heating to 60° C. was initiated. Once at this temperature, Zinc Oxide; Triethoxycaprylylsilane was slowly added to the beaker and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Cooling to 40-45° C. was initiated. Once at this temperature, Ascorbyl Palmitate; Tocopherol; Lecithin and Tocopheryl Acetate were added and mixed until homogenous. Mixing with the propeller was stopped and the formula was homogenized with a SILVERSON Benchtop Homogenizer at 4,000 RPM for 2.5 minutes.

For Comparative Composition E, Zinc Oxide; Triethoxycaprylylsilane was slowly added to the beaker at room temperature and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Mixing with the propeller was stopped and the formula was homogenized with a SILVERSON Benchtop Homogenizer at 4,000 RPM for 2.5 minutes. The beaker was returned to the hotplate and the propeller was placed back into the solution to re-initiate mixing. Heating to 85-90° C. was initiated. Once at ˜70° C., Polyamide-8 was added to the beaker. The batch was mixed for 10 minutes at 85-90° C. The formula was cooled to 40° C. under mixing. Once at 40° C. the batch was completed and removed from the hotplate and propeller.

For Comparative Composition F, heating to 70-75° C. was initiated. Once at this temperature, Triacontanyl PVP was added and mixed until dispersed into the solution. A check was performed to ensure there were no undispersed particles in the glass beaker. Next Zinc Oxide; Triethoxycaprylylsilane was slowly added to the beaker and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Afterwards, cooling to 35-40° C. was initiated. Once at this temperature, mixing with the propeller was stopped and the formula was homogenized with a SILVERSON Benchtop Homogenizer at 4,000 RPM for 2 minutes.

Comparative Compositions A-F were tested for stability according to the parameters of Table 1. They were not stable. The details are shown in Table 4.

TABLE 4

| Comparative | | |
|---|---|---|
| Example | Separation | Sedimentation |
| A | RT sample had | RT sample had |
| | full separation | sedimentation |
| B | No separation | RT sample had |
| | observed | sedimentation |
| C | No separation | RT sample had |
| | observed | sedimentation |
| D | No separation | RT sample had |
| | observed | sedimentation |
| E | RT had ~2 inch | RT sample had |
| | separation | sedimentation |
| F | No separation | RT sample had |
| | observed | sedimentation |

### Example 3

The following Comparative Compositions G-L were made using the ingredients shown in Table 5.

In vitro SPF was tested on Comparative Compositions I-L using the IN VITRO SPF TEST METHOD set forth above. The results are also shown in Table 5.

TABLE 5

| | | Weight (%) | | | | | |
|---|---|---|---|---|---|---|---|
| INCI Name | Function | G | H | I | J | K | L |
| Helianthus Annuus | Carrier Oil | 0 | 0 | 57.5 | 73.15 | 55.5 | 55.5 |
| (Sunflower Seed | | | | | | | |
| Oil) | | | | | | | |
| C12-15 Alkyl | Carrier Oil | 58.5 | 61 | 0 | 0 | 0 | 0 |
| Benzoate | | | | | | | |
| Isododecane | Emollient | 8 | 8 | 12 | 0 | 8 | 8 |
| Diisopropyl Adipate | Emollient | 5 | 5 | 3 | 0 | 5 | 5 |
| VP/Hexadecene | Film Former | 2.5 | 0 | 0 | 0 | 0 | 2.5 |
| Copolymer | | | | | | | |
| Triacontanyl PVP | Film Former | 0 | 0 | 0 | 0 | 2.5 | 0 |
| Polyester-7 (and) | Film Former | 0 | 0 | 0 | 0 | 2.5 | 2.5 |
| Neopentyl Glycol | | | | | | | |
| Diheptanoate | | | | | | | |
| Polyamide-8 | Film Former | 0 | 0 | 1 | 0 | 0 | 0 |
| Poly C10-30 Alkyl | Film Former | 0 | 0 | 0 | 0.35 | 0 | 0 |
| Acrylate | | | | | | | |
| Tocopheryl Acetate | Antioxidant | 0.5 | 0.5 | 0 | 0 | 0 | 0 |
| Polyhydroxystearic | Dispersing | 1.5 | 1.5 | 1.5 | 1.5 | 1.5 | 1.5 |
| Acid | Agent | | | | | | |
| Zinc Oxide; | Sunscreen | 12 | 12 | 10 | 10 | 10 | 10 |
| Triethoxycaprylylsilane | Agent | | | | | | |
| Titanium Dioxide; | Sunscreen | 12 | 12 | 15 | 15 | 15 | 15 |
| Aluminum | Agent | | | | | | |
| Hydroxide; Stearic | | | | | | | |
| Acid | | | | | | | |

| In vitro SPF | Not | Not | 15.79 | 12.896 | 119.97 | 20.84 |
|---|---|---|---|---|---|---|
| | tested | tested | | | | |

Comparative Compositions G-L were prepared by adding the carrier oil to a glass beaker at room temperature and placing that beaker on a hot plate. A propeller was lowered into the beaker and turned on to such a speed to form a vortex. To the glass beaker Isododecane and Diisopropyl Adipate were added (with the exception of Comparative Composition J). After the emollients were incorporated into solution, Polyhydroxystearic Acid was added to the glass beaker and mixed well.

For Comparative Composition G, Tocopheryl Acetate was added to the beaker and mixed until homogenous. Next, Zinc Oxide; Triethoxycaprylylsilane was slowly added to the beaker at room temperature and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Mixing with the propeller was stopped and the formula was homogenized with a SILVERSON Benchtop Homogenizer at 4,000 RPM for 2.5 minutes.

For Comparative Composition H Zinc Oxide; Triethoxycaprylylsilane was slowly added to the beaker at room temperature and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Tocopheryl Acetate was then added to the beaker and mixed until homogenous. Mixing with the propeller was stopped and the formula was homogenized with a SILVERSON Benchtop Homogenizer at 4,000 RPM for 2.5 minutes.

For Comparative Composition I, Zinc Oxide; Triethoxycaprylylsilane was slowly added to the beaker at room temperature and was mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Mixing with the propeller was stopped and the formula was homogenized with a SILVERSON Benchtop Homogenizer at 4,000 RPM for 2.5 minutes. The beaker was returned to the hotplate and the propeller was placed back into the solution to re-initiate mixing. Heating to 89° C. was initiated. Once at ˜70° C., Polyamide-8 was added to the beaker. The batch was mixed for 10 minutes at 89° C. Cooling to 35-40° C. was initiated. At 65° C., mixing with the propeller was stopped and the formula was homogenized with a SILVERSON Benchtop Homogenizer at 4,000 RPM for 1 minute. The beaker was returned to the hotplate and the propeller was placed back into the solution to re-initiate mixing. Once temperature of the batch reached 35-40° C., mixing with the propeller was stopped and the formula was homogenized with a SILVERSON Benchtop Homogenizer at 4,000 RPM for 1 minute.

For Comparative Composition J, Zinc Oxide; Triethoxycaprylylsilane was slowly added to the beaker at room temperature and was mixed for 5 minutes to ensure the mineral filter was well dispersed. Then Titanium Dioxide; Aluminum Hydroxide; Stearic Acid was added to the beaker and was mixed for 5 minutes to ensure the mineral filter was well dispersed. Mixing with the propeller was stopped and the formula was homogenized with SILVERSON Benchtop Homogenizer at 4,000 RPM for 2 minutes. The beaker was returned to the hotplate and the propeller was placed back into the solution to re-initiate mixing. Heating to 80° C. was initiated. While heating, Poly C10-30 Alkyl Acrylate was added to the beaker. The batch was mixed at temperature (80° C.) for 5-10 minutes. The formula was cooled to 40° C. under mixing. Once at 40° C. the batch was completed and removed from the hotplate and propeller.

For Comparative Composition K, Polyester-7 (and) Neopentyl Glycol Diheptanoate was added to the beaker and mixed until homogenous. Heating to 70° C. was initiated. While heating, Triacontanyl PVP was added to the batch. Zinc Oxide; Triethoxycaprylylsilane was slowly added to the beaker at room temperature and was mixed in for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Cooling to room temperature was initiated. After the batch reached 30-40° C., mixing with the propeller was stopped and the formula was homogenized with a SILVERSON Benchtop Homogenizer at 4,000 RPM for 2 minutes.

For Comparative Composition L, Polyester-7 (and) Neopentyl Glycol Diheptanoate and VP/Hexadecene was added to the beaker and mixed until homogenous. Next Zinc Oxide; Triethoxycaprylylsilane was slowly added to the beaker at room temperature and was mixed for 5 minutes to ensure the mineral filter was well dispersed. Next Titanium Dioxide; Aluminum Hydroxide; Stearic Acid was added to the beaker and mixed for 5 minutes to ensure the mineral filter was well dispersed. Mixing with the propeller was stopped and the formula was homogenized with a SILVERSON Benchtop Homogenizer at 4,000 RPM for 2 minutes.

Comparative Compositions G-L were tested for stability according to the parameters of Table 1. They were not stable. The details are shown in Table 6.

TABLE 6

| Comparative | | |
|---|---|---|
| Composition | Separation | Sedimentation |
| G | RT had full | No sedimentation |
| | separation | observed |
| H | RT had full | RT sample had |
| | separation | sedimentation |
| I | RT sample had ~2 | RT sample had |
| | inch separation | sedimentation |
| J | RT had ~3 inch | RT sample had |
| | separation | sedimentation |
| K | No separation | RT sample had |
| | observed | sedimentation |
| L | No separation | RT sample had |
| | observed | sedimentation |

### Example 4

The following Comparative Compositions M-R were made using the ingredients shown in Table 7.

In vitro SPF was tested on Comparative Compositions M, P, Q, and R using the IN VITRO SPF TEST METHOD set forth above. The results are also shown in Table 7.

TABLE 7

| | | Weight (%) | | | | | |
|---|---|---|---|---|---|---|---|
| INCI Name | Function | M | N | O | P | Q | R |
| Helianthus Annuus | Carrier Oil | 59 | 30.5 | 0 | 56 | 44 | 72 |
| (Sunflower Seed Oil) | | | | | | | |
| Caprylic/Capric | Carrier Oil | 0 | 30.5 | 0 | 0 | 0 | 0 |
| Triglyceride | | | | | | | |
| C12-15 Alkyl | Carrier Oil | 0 | 0 | 56 | 0 | 0 | 0 |
| Benzoate | | | | | | | |
| Isododecane | Emollient | 12 | 8 | 8 | 8 | 12 | 0 |
| Diisopropyl Adipate | Emollient | 3 | 5 | 5 | 5 | 3 | 0 |
| VP/Hexadecene | Film Former | 0 | 0 | 2.5 | 2.5 | 0 | 0 |
| Copolymer | | | | | | | |
| Triacontanyl PVP | Film Former | 0 | 0 | 2.5 | 0 | 0 | 0 |
| Octyldodecyl Citrate | Film Former | 0 | 0 | 0 | 2.5 | 0 | 0 |
| Crosspolymer | | | | | | | |
| Tocopheryl Acetate | Antioxidant | 0 | 0.5 | 0.5 | 0.5 | 0 | 0 |
| Polyamide-8 | Film Former | 0 | 0 | 0 | 0 | 15 | 0 |
| Poly C10-30 Alkyl | Film Former | 0 | 0 | 0 | 0 | 0 | 1.5 |
| Acrylate | | | | | | | |
| Polyhydroxystearic | Dispersing | 1 | 1.5 | 1.5 | 1.5 | 1 | 1.5 |
| Acid | Agent | | | | | | |
| Zinc Oxide; | Sunscreen | 10 | 12 | 12 | 12 | 10 | 10 |
| Triethoxycaprylylsilane | Agent | | | | | | |
| Titanium Dioxide; | Sunscreen | 15 | 12 | 12 | 12 | 15 | 15 |
| Aluminum | Agent | | | | | | |
| Hydroxide; Stearic | | | | | | | |
| Acid | | | | | | | |

| | | | | | | |
|---|---|---|---|---|---|---|
| In vitro SPF | 17.72 | Not | Not | 23.756 | 53.9 | 21.664 |

| | |
|---|---|
| Tested | Tested |

Comparative Compositions M-R were prepared by adding the carrier oil to a glass beaker at room temperature and placing that beaker on a hot plate. A propeller was lowered into the beaker and turned on to such a speed to form a vortex. To the glass beaker Isododecane and Diisopropyl Adipate (with the exception of comparative example R) were added. After the emollients were incorporated into solution, Polyhydroxystearic Acid was added to the glass beaker and mixed well.

For Comparative Compositions M and N, Zinc Oxide; Triethoxycaprylylsilane was then slowly added to the beaker at room temperature and was mixed for 5 minutes to ensure the mineral filter was well dispersed. Next Titanium Dioxide; Aluminum Hydroxide; Stearic Acid was added to the beaker and mixed for 5 minutes to ensure the mineral filter was well dispersed. The remainder of the ingredients, antioxidants and fragrance (if applicable) were added to beaker and mixed for 5 minutes. Then mixing with the propeller was stopped and the formula was homogenized with a SILVERSON Benchtop Homogenizer at 3,500-4,000 RPM for 2-4 minutes depending on the batch size.

For Comparative Composition O heating to 70-75° C. was then initiated. Once at this temperature, Triacontanyl PVP was added and mixed in until dispersed into the solution. A check was performed to ensure there were no undispersed particles in the glass beaker. Then cooling to 35-40° C. began. While the solution was cooling, Zinc Oxide; Triethoxycaprylylsilane was slowly added to the beaker and was mixed in for 5 minutes to ensure the mineral filter was well dispersed. Then Titanium Dioxide; Aluminum Hydroxide; Stearic Acid was added to the beaker and was mixed for 5 minutes to ensure the mineral filter was well dispersed. Once the solution reached 35-40° C., the remainder of the ingredients, antioxidants and fragrance (if applicable) were added to beaker and mixed for 5 minutes. Mixing with the propeller was stopped and the formula was homogenized with a SILVERSON Benchtop Homogenizer at 3,500-4,000 RPM for 2-4 minutes depending on the batch size.

For Comparative Composition P, while mixing VP/Hexadecene Copolymer was added to the glass beaker. Heating was then initiated to 80° C. While heating, Octyldodecyl Citrate Crosspolymer was added to the beaker and mixed until incorporated into solution. Zinc Oxide; Triethoxycaprylylsilane was then slowly added to the beaker and was mixed for 5 minutes to ensure the mineral filter was well dispersed. Then Titanium Dioxide; Aluminum Hydroxide; Stearic Acid was added to the beaker and was mixed for 5 minutes to ensure the mineral filter was well dispersed. Cooling to 40-45° C. began. Once at this temperature, Tocopheryl Acetate was added and mixed until homogenous. Mixing with the propeller was stopped and the formula was homogenized with a SILVERSON Benchtop Homogenizer at 3,500-4,000 RPM for 2-4 minutes depending on the batch size.

For Comparative Composition Q, Zinc Oxide; Triethoxycaprylylsilane was slowly added to the beaker at room temperature and was mixed in for 5 minutes to ensure the mineral filter was well dispersed. After 5 minutes of mixing, Titanium Dioxide; Aluminum Hydroxide; Stearic Acid was added to the beaker and mixed for 5 minutes to ensure the mineral filter was well dispersed. Mixing with the propeller was then stopped and the formula was homogenized with a SILVERSON Benchtop Homogenizer at 4,000 RPM for 2.5 minutes. The beaker was returned to the hotplate and the propeller was placed back into the solution to re-initiate mixing. Heating to 85-90° C. was initiated. Once at ˜70° C., Polyamide-8 was added to the beaker. The batch was mixed for 15 minutes at 85-90° C. The formula was then cooled to 40° C. under mixing. Once at 40° C. the batch was completed and removed from the hotplate and propeller.

For Comparative Composition R, Zinc Oxide; Triethoxycaprylylsilane was slowly added to the beaker at room temperature and mixed for 5 minutes to ensure the mineral filter was well dispersed. Next Titanium Dioxide; Aluminum Hydroxide; Stearic Acid was added to the beaker and was mixed for 5 minutes to ensure the mineral filter was well dispersed. Mixing with the propeller was stopped and the formula was homogenized with a SILVERSON Benchtop Homogenizer at 4,000 RPM for 2 minutes. The beaker was returned to the hotplate and the propeller was placed back into the solution to re-initiate mixing. Heating to 82° C. was initiated. While heating, Poly C10-30 Alkyl Acrylate was added to the beaker. The batch was mixed at temperature (82° C.) for 10 minutes. The formula was then cooled to 40° C. under mixing. Once at 40° C. the batch was completed and removed from the hotplate and propeller.

The compositions were tested for stability according to the parameters of Table 1. The details are shown in Table 8.

Comparative Compositions Q and R exhibited stability at room temperature.

Comparative Compositions M, N, O, and P were not stable at room temperature.

TABLE 8

| Composition | Separation | Sedimentation |
|---|---|---|
| M | No separation | RT sample had |
| | observed | sedimentation |
| N | No separation | RT sample had |
| | observed | sedimentation |
| O | RT sample had | No sedimentation |
| | full separation | observed |
| P | No separation | RT had |
| | observed | sedimentation |
| Q | No separation | No sedimentation |
| | observed | observed |
| R | No separation | No sedimentation |
| | observed | observed |

### Example 5

Compositions E1, E2, E3, and E4 according to the invention and Comparative Compositions Q and R were measured for shear viscosity and tested for sprayability.

Shear viscosity was measured using the SHEAR VISCOSITY TEST METHOD.

Sprayability of each composition was tested by shaking 5-10 times in a glass jar. After shaking the sample, the composition was transferred to a bottle fitted with a new Mark VII Max-20/410-WT25 Insert pump supplied by Silgan. After the pump was inserted into the composition, the pump spray head was pressed down until composition was drawn up and primed the pump. Additional pumping evacuated the pump head. If the composition was unable to evacuate out of the pump head, the sample was deemed not sprayable.

The results are shown in Table 9.

TABLE 9

| Test | Shear Viscosity | |
|---|---|---|
| Composition | η at 1,000 s⁻¹ (cP) | Sprayability |
| E1 | 177 | Yes |
| E2 | 129 | Yes |
| E3 | 92.6 | Yes |
| E4 | 91.1 | Yes |
| Q | 763 | No |
| R | 324 | No |

Compositions E1-E4 according to the invention had shear viscosities of 91.1 to 177 cP at 1,000 s⁻¹. Consistent with that, Compositions E1-E4 were sprayable and required little effort to spray.

In contrast, Comparative Compositions Q and R having shear viscosities of 763 and 324 cP at 1,000 s⁻¹, respectively, were not sprayable.

1. An anhydrous sunscreen composition comprising:
(a) at least about 15% by weight based on the total weight of the sunscreen composition of a combination of titanium dioxide and zinc oxide, wherein the weight ratio of zinc oxide to titanium dioxide in the composition is about 0.5 to about 1.5;
(b) at least about 50% by weight based on the total weight of the sunscreen composition of a carrier oil, wherein the carrier oil comprises at least 50% by weight Helianthus Annuus (Sunflower) Seed Oil;
(c) about 10% to about 20% by weight based on the total weight of the sunscreen composition a combination of Isododecane and Diisopropyl Adipate; and
(d) about 2% to about 6% by weight based on the total weight of the sunscreen composition of a film former comprising triacontanyl PVP.

2. The anhydrous sunscreen composition of claim 1, wherein the carrier oil comprises at least 80% by weight Helianthus Annuus (Sunflower) Seed Oil.

3. The anhydrous sunscreen composition of claim 1, wherein the carrier oil further comprises Caprylic/Capric Triglyceride.

4. The anhydrous sunscreen composition of claim 1, wherein the film former further comprises VP/Hexadecene Copolymer.

5. The anhydrous sunscreen composition of claim 1, further comprising Polyhydroxystearic Acid.

6. The anhydrous sunscreen composition of claim 1, wherein the sunscreen composition is substantially free of polyester-7.

7. The anhydrous sunscreen composition of claim 1 comprising at least about 24% by weight based on the total weight of the sunscreen composition of the combination of titanium dioxide and zinc oxide.

8. The anhydrous sunscreen composition of claim 1, wherein the weight ratio of zinc oxide to titanium dioxide in the composition is about 0.67 to about 1.4.

9. The anhydrous sunscreen composition of claim 1 comprising about 13% by weight based on the total weight of the sunscreen composition the combination of Isododecane and Diisopropyl Adipate.

10. The anhydrous sunscreen composition of claim 1 comprising about 2.5% to about 3.2% by weight based on the total weight of the sunscreen composition of triacontanyl PVP and about 0.5% to about 2.5% by weight based on the total weight of the sunscreen composition of VP/Hexadecene Copolymer.

11. The anhydrous sunscreen composition of claim 1 having a shear viscosity of about 75 to about 200 cP at 1,000 s⁻¹ at 25° C.
